(19) United States
(12) Patent Application Publication
(10) Pub. No.: US 2009/0311707 A1
(43) Pub. Date: Dec. 17, 2009

(54) O-GLYCANS AS DIAGNOSTIC MARKERS FOR INFLAMMATORY BOWEL DISEASE

(76) Inventor: LIJUN XIA, Edmond, OK (US)

Correspondence Address: FULBRIGHT & JAWORSKI L.L.P., 600 CONGRESS AVE., SUITE 2400, AUSTIN, TX, 78701, US

(21) Appl. No.: 12/486,262

(22) Filed: Jun. 17, 2009

Related U.S. Application Data
(60) Provisional application No. 61/073,170, filed on Jun. 17, 2008.

(51) Int. Cl. C12Q 1/68; G01N 33/68; C12N 1/02; G01N 33/566; C40B 30/04

(52) U.S. Cl. 435/6; 436/87; 435/29; 436/501; 506/9

(57) ABSTRACT

The present invention provides diagnostic methods for inflammatory bowel disorders (e.g., Crohn's disease or ulcerative colitis) comprising assessing expression, structure and/or function of O-glycans in a sample from a subject, as well as antibodies to such molecules.

### PRIORITY CLAIM

The present application claims benefit of priority to U.S. Ser. No. 61/073,170, filed Jun. 17, 2008, the entire contents of which are hereby incorporated by reference.

### GOVERNMENT SUPPORT CLAUSE

This invention was made with government support under grant number P20-RR018758 from the National Institutes of Health and grant support from Crohn's and Colitis Foundation of America (CCFA Ref. #1612). The government has certain rights in the invention.

### BACKGROUND

1. Field of the Invention

The present invention relates generally to the fields of glycobiology and medicine. More particularly, it concerns assessing O-glycan expression, structure of function in a subject to identify or predict inflammatory bowel diseases (e.g., Crohn's disease and ulcerative colitis), and colorectal cancer.

2. Description of Related Art

Ulcerative colitis is a common form of inflammatory bowel diseases (IBD). It is generally recognized as an immune-mediated disorder resulting from an abnormal interaction between colonic microflora and mucosal immune cells in a genetically susceptible host (Sartor, 2003; Podolsky, 2002; Elson et al., 2003). The nature of the mucosal immune abnormality remains unclear, and how this interaction is allowed to develop is not well understood.

Traditional diagnosis of IBD usually starts with assessing symptomatic aspects of the disease, including abdominal pain, vomiting, diarrhea, hematochezia, weight loss and various associated complaints or diseases (arthritis, pyoderma gangrenosum, primary sclerosing cholangitis). Clearly, these symptoms overlap with numerous other conditions. Thus, a confident diagnosis generally requires colonoscopy with biopsy of pathological lesions. Non-invasive techniques are therefore very much needed.

Altered intestinal O-glycan expression has long been observed in patients with IBD and colorectal cancer (Corfield et al., 2001; Rhodes, 1997; Podolsky and Isselbacher, 1984). Mice lacking intestinal O-glycans develop colitis and colorectal tumors (An et al, 2007), supporting a causal role for abnormal O-glycans in the pathogenesis of these diseases. However, to date, the ability to examine O-glycans in a subject and extrapolate to a diagnostic determination has not been achieved.

### SUMMARY OF THE INVENTION

Thus, in accordance with the present invention, there is provided a method of diagnosing or predicting inflammatory bowel disease (IBD) in a subject comprising (a) obtaining a sample comprising mucins from the subject; (b) assessing mucin-related O-glycan structure and/or expression in the sample; (c) diagnosing or predicting IBD in the subject where mucin-related O-glycan structure or expression is abnormal. The sample may be a fecal sample, a colorectal swab or a colon lavage. Alternatively, the sample may be a colon tissue sample. The IBD may be ulcerative colitis or Crohn's Disease.

O-glycan structure, such as a core-1 glycan structure, a core-3 glycan structure, or both may be assessed. Alternatively, or in addition, O-glycan expression may be assessed. Assessing may comprise immunological recognition of the O-glycan structure, e.g., of T, Tn, sialyl-T and/or sialyl-Tn, such as by ELISA or Western blot. Assessing may also comprise lectin or antibody array analysis.

The method may further comprising obtaining other diagnostic information from the subject, such as one or more of a patient physical, a patient medical history, a family medical history, an CBC, a serological antibody screen for anti-CBir1, anti-OmpC, or anti-pANCA, a colonoscopy, a sigmoidoscopy, leukocyte scintigraph, or colon x-ray.

In another embodiment, there is provided a method of diagnosing or predicting inflammatory bowel disease (IBD) in a subject comprising (a) obtaining a tissue sample from the subject; (b) assessing the structure and/or transcription of one or more genes involved in mucin-related O-glycan trafficking, stability, synthesis or processing in a cell of the sample; (c) diagnosing or predicting IBD in the subject where one or more of the genes involved in mucin-related O-glycan trafficking, stability synthesis or processing is altered in structure or transcription. The tissue sample may be a colon tissue sample, or a tissue that comprises endothelial cells, epithelial cells and/or hematopoietic cells. The IBD may be ulcerative colitis or Crohn's Disease.

The gene may be one that encodes a protein involved in mucin-related O-glycan synthesis, such as T-synthase, C2Gnt, or C3Gnt. The gene may encode a protein involved in mucin-related O-glycan processing, or the gene may encode a protein involved in mucin-related O-glycan trafficking or stability, such as Cosmc. Assessing may comprise RT-PCR, sequencing, primer extension, Northern blot or gene array analysis.

The method may further comprise obtaining other diagnostic information from the subject, such as one or more of a patient physical, a patient medical history, a family medical history, an CBC, a serological antibody screen for anti-CBir1, anti-OmpC, or anti-pANCA, a colonoscopy, a sigmoidoscopy, leukocyte scintigraph, or colon x-ray.

In yet another embodiment, there is provided a method of diagnosing or predicting inflammatory bowel disease (IBD) in a subject comprising (a) obtaining a tissue sample from the subject; (b) assessing the activity or level of one or more proteins involved in mucin-related O-glycan trafficking, stability, synthesis or processing in a cell of the sample; (c) diagnosing or predicting IBD in the subject where one or more of the genes involved in mucin-related O-glycan trafficking, stability synthesis or processing is altered in activity or level. The tissue sample may be a colon tissue sample, or tissue sample that comprises endothelial cells, epithelial cells and/or hematopoietic cells. The IBD may be ulcerative colitis or Crohn's Disease.

The protein may be involved in mucin-related O-glycan synthesis, such as T-synthase, C2Gnt or C3Gnt. The protein may be involved in mucin-related O-glycan processing, or may be involved in mucin-related O-glycan trafficking or stability, such as Cosmc. Assessing may comprise an enzyme assay for, or immunologic detection of, the one or more proteins.

The method may further comprise obtaining other diagnostic information from the subject, such as one or more of a patient physical, a patient medical history, a family medical history, an CBC, a serological antibody screen for anti-CBir1, anti-OmpC, or anti-pANCA, a colonoscopy, a sigmoidoscopy, leukocyte scintigraph, or colon x-ray.

In still a further embodiment, there is provided a method of diagnosing or predicting inflammatory bowel disease (IBD) in a subject comprising (a) obtaining a sample comprising antibodies from the subject; (b) assessing O-glycan-binding antibodies in the sample; (c) diagnosing or predicting IBD in the subject where O-glycan-binding antibodies in the sample are abnormal. The sample may be serum. The IBD may be ulcerative colitis or Crohn's Disease.

Assessing may comprise measuring antibody level, antibody specificity, or both. The specificity may be for T, Tn, sialyl-T and/or sialyl-Tn. The method may further comprise obtaining other diagnostic information from the subject, such as one or more of a patient physical, a patient medical history, a family medical history, an CBC, a serological antibody screen for anti-CBir1, anti-OmpC, or anti-pANCA, a colonoscopy, a sigmoidoscopy, leukocyte scintigraph, or colon x-ray.

In still yet another embodiment, there is provided a method of classifying inflammatory bowel disease (IBD) in a subject comprising (a) obtaining a sample comprising mucins from the subject; (b) assessing mucin-related O-glycan structure and/or expression in the sample; (c) classifying the IBD in the subject based on the amount of mucin-related O-glycan expression, as compared to a normal and/or abnormal standard. Classifying may comprise determining whether the IBD is active or inactive. The O-glycan may be Tn antigen. Assessing may comprise Western blot or lectin binding. The IBD may be ulcerative colitis. The sample may be a colon lavage.

In still yet an additional embodiment, there is provided a method of monitoring disease progression or therapeutic efficacy in a subject with inflammatory bowel disease (IBD) comprising (a) obtaining a first sample comprising mucins from the subject; (b) assessing mucin-related O-glycan structure and/or expression in the first sample; (c) obtaining a second sample comprising mucins from the subject at a later point in time as compared to the first sample; (d) assessing mucin-related O-glycan structure and/or expression in the second sample; (e) comparing mucin-related O-glycan structure and/or expression between the first and second samples.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-B—PAS (Periodic Acid-Schiff) staining of glycans in human colon lavage. (FIG. 1A) PBS+TX: Processed samples (initial extracts acetone precipitated followed by extraction of pellet with 9M urea+2% CHAPS). (FIG. 1B) V9: Unprocessed samples. Mucin (porcine stomach mucin; Sigma) was used as a positive control, whereas Sigma albumin was a negative control. Samples were reduced with 2-mercaptoethanol by boiling 10 min and then separated on 10% SDS-PAGE for 2 hrs at 150 V. PAS staining was done according to standard protocols.

FIGS. 2A-C—Western blot analysis of Tn antigen in human colon lavages. (FIG. 2A) Blot of PBS+TX samples (processed samples: initial extracts acetone precipitated followed by extraction of pellet with 9M urea+2% CHAPS) was probed with HPA lectin, which recognizes the non-sialylated Tn antigen. Mucins (porcine stomach mucins, Sigma) and albumin were used as controls. (FIG. 2B) Blot of V9 samples (unprocessed samples) was probed with HPA lectin. (FIG. 2C) Blot of PBS+TX samples was detected by anti-Tn IgM mAb. BSM (bovine submaxillary mucin, Sigma), which contains Tn antigen, was used as a positive control.

FIG. 3—Colonic epithelial cells of patients with UC express Tn antigen. Sections of human colon biopsy samples were stained an anti-Tn mAb. Brown color indicates positive staining.

FIG. 4—Colonic epithelial cells of patients with Crohn's express Tn antigen. Sections of human colon biopsy samples were stained an anti-Tn mAb. Brown color indicates positive staining.

### DESCRIPTION OF ILLUSTRATIVE EMBODIMENTS

O-glycans are primary components of the intestinal mucus gel layer that overlies the gut epithelium. This layer is a dense polysaccharide-rich matrix that, together with epithelial cells, composes the intestinal barrier, which functions to prevent intestinal microflora from encountering intestinal mucosal immune cells. The mucus layer consists primarily of mucins, molecules rich in serine and threonine to which O-linked oligosaccharides (O-glycans) are frequently attached. Over 80% of mucin mass consists of O-glycans. O-glycans have two main subtypes referred to as core 1- and core 3-derived O-glycans, and the biosynthesis of these subtypes is controlled by specific glycosyltransferases.

In previous studies, in an effort to address the role of O-glycans in IBD and GI cancer in vivo, the inventors established a mouse line that is deficient in core1-derived O-glycans. They also have also developed a line that is deficient in core 3-derived O-glycans, and a line that is deficient in both. By using these mouse lines, the inventors were able to identify specific contributions to the aforementioned disease states and, moreover, to alleviate symptoms of these disease states by administration of O-glycans to subjects.

The present invention extends the inventor's work and demonstrates that certain O-glycans can indicate the presence or likelihood of IBD. Thus, the inventor now provides diagnostic and prognostic methods that examine O-glycan structure, expression and function, as well as antibodies directed to O-glycans.

### I. O-GLYCANS

Glycoproteins with O-glycosidically linked carbohydrate chains of complex structures and functions are found in secretions and on the cell surfaces of cancer cells. The structures of O-glycans are often unusual or abnormal in cancer, and greatly contribute to the phenotype and biology of cancer cells. Some of the mechanisms of changes in O-glycosylation pathways have been determined in cancer model systems. However, O-glycan biosynthesis is a complex process. The glycosyltransferases that synthesize O-glycans appear to exist as families of related enzymes of which individual members are expressed in a tissue- and growth-specific fashion. Studies of their regulation in cancer may reveal the connection between cancerous transformation and glycosylation which may help to understand and control the abnormal biology of tumor cells. Cancer diagnosis may be based on the appearance of certain glycosylated epitopes, and therapeutic avenues have been designed to attack cancer cells via their glycans.

A. Mucins

Mucins are high-molecular weight epithelial glycoproteins with a high content of clustered oligosaccharides O-glycosidically linked to tandem repeat peptides rich in threoiine, serine, and proline. There are two structurally and functionally distinct classes of mucins: secreted gel-forming mucins (MUC2, MUC5AC, MUC5B, and MUC6) and transmembrane mucins (MUC1, MUC3A, MUC3B, MUC4, MUC 12, MUC 17), although the products of some MUC genes do not fit well into either class (MUC7, MUC8, MUC9, MUC13, MUC15, MUC16). MUC1 mucin, as detected immunologically, is increased in expression in colon cancers, which correlates with a worse prognosis. Expression of MUC2 secreted gel-forming mucin is generally decreased in colorectal adenocarcinoma, but preserved in mucinous carcinomas, a distinct subtype of colon cancer associated with microsatellite instability. Another secreted gel-forming mucin, MUC5AC, a product of normal gastric mucosa, is absent from normal colon, but frequently present in colorectal adenomas and colon cancers. The O-glycosidically linked oligosaccharides of mucins can be described in terms of core type, backbone type, and peripheral structures.

B. O-Glycan Structure and Synthesis

O-glycans are synthesized post-translationally in the Golgi apparatus through a series of sequential reactions catalyzed by specific glycosyltransferases. The primary structure of O-glycans is known as Tn antigen (GalNAcα-O-Ser/Thr), the only structure common to all mucin-type O-glycans. Tn antigen is normally modified by additional glycosylations, O-glycans have two main subtypes, classified as core 1- and core 3-derived structures. O-glycans consist larged of core 1 and core 2 O-glycans, with core 1 predominating. Core 1 is formed by adding galactose (Gal) to the N-acetylgalactosamine (GalNAc), a function performed solely by the enzyme core 1 β1,3-galactosylgransferase (T-synthase). Core 1 can be further branched to form the core 2 structure, and this branching is catalyzed by three different core 2 glycosyltransferases. The production of the core 3-derived O-glycans is controlled by core 3 β1,3-N-acetylglucosaminyltransferase (C3GnT), which uses the same GalNAcαc-Ser/Thr substrate as T-synthase. Core 1-derived O-glycans are present in most tissues, and are especially plentiful in endothelial cells, epithelial cells, and hematopoietic cells. The expression of core 3-derived O-glycans is restricted to the colon and salivary glands. The biosynthesis of O-glycans may be terminated, as is the case during production of T or Tn antigens in the absence of core 2 glycosyltransferases and/or T-synthase expression. O-glycans may also be modified in a number of ways by sialylation, fucosylation, and sulfation. For example, certain sialyltransferases can modify the T or Tn antigen to sialyl T or sialyl Tn antigens.

C. O-Glycan Purification

O-glycans of the present invention may be purified from a sample. Generally, for purification, the inventors will follow published methods for purification of mucins from fresh porcine stomach or colon with modifications (Xia et al., 2005; Feste et al., 1990). Briefly, rinsing in water, the mucosal layer (including epithelium and mucus) is homogenized in ice-cold water (˜1 part mucosa: 1 part water, final slurry), and centrifuged to remove insoluble debris. The soluble mucins in the supernatant are precipitated by adjusting to pH 5.0 with 100 mM HCl followed by centrifugation (10,000×g, 4° C., 10 min). The pellet is resolubilized and adjusted to pH 7.2 with 100 mM NaOH, then extracted twice in methanol:chloroform (1:1 v/v) prior to a second centrifugation. The middle phase is collected and dialyzed (12-14,000 MWCO) followed by sequential treatment with heparinum Heparinase II (0.075 U/ml, Sigma), chondroitinase ABC (0.015 U/ml, Sigma), DNase (75 U/ml, Invitrogen), RNase (0.01 mg/ml, Invitrogen), and proteinase K (0.25 U/ml, O/N at 65° C., Sigma). These treatments eliminate contaminating lipids, polypeptides, and nucleotides. The mucin is then collected as a >200 kDa void volume fraction by size exclusion chromatography (Sephacryl HR-S-200, Pharmacia) in isotonic buffer (50 mM Tris, 100 mM NaCl, pH 7.4). The void volume fraction is dialyzed, lyophilized, weighed, and stored at −80° C. The quality of the purified mucins is verified by SDS-PAGE using a 3% stacking and a 4% separating gel that is stained by PAS. Protein will be measured using a BCA kit (Pierce).

### II. INFLAMMATORY BOWEL DISEASE

The methods of the present invention address diagnosis and/or prediction of an inflammatory bowel disease. The term “inflammatory bowel disease” or “IBD,” as used herein, describes a broad class of diseases characterized by inflammation of at least part of the gastrointestinal tract. IBD symptoms may include inflammation of the intestine and resulting in abdominal cramping and persistent diarrhea. Inflammatory bowel diseases include ulcerative colitis (UC), Crohn's disease (CD), indeterminate colitis, chronic colitis, discontinuous or patchy disease, ileal inflammation, extracolonic inflammation, granulomatous inflammation in response to ruptured crypts, aphthous ulcers, transmural inflammation, microscopic colitis, diverticulitis and diversion colitis.

A. Ulcerative Colitis

As discussed above, altered intestinal O-glycan expression has long been observed in patients with IBD, such as ulcerative colitis, but the role of this alteration in the etiology of these diseases is unknown (Rhodes, 1996; 1997; Podolsky & Fournier, 1988). Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis. The inflammation makes the colon empty frequently, causing diarrhea. Ulcers form in places where the inflammation has killed the cells lining the colon; the ulcers bleed and produce pus.

Ulcerative colitis may occur in people of any age, but most often it starts between ages 15 and 30, or less frequently between ages 50 and 70. Children and adolescents sometimes develop the disease. Ulcerative colitis affects men and women equally and appears to run in some families. Theories about what causes ulcerative colitis abound, but none have been proven. The most popular theory is that the body's immune system reacts to a virus or a bacterium by causing ongoing inflammation in the intestinal wall. People with ulcerative colitis have abnormalities of the immune system, but doctors do not know whether these abnormalities are a cause or a result of the disease. Ulcerative colitis is not caused by emotional distress or sensitivity to certain foods or food products, but these factors may trigger symptoms in some people.

The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

A thorough physical exam and a series of tests may be required to diagnose ulcerative colitis. Blood tests may be done to check for anemia, which could indicate bleeding in the colon or rectum. Blood tests may also uncover a high white blood cell count, which is a sign of inflammation somewhere in the body. By testing a stool sample, the doctor can detect bleeding or infection in the colon or rectum. The doctor may do a colonoscopy or sigmoidoscopy. For either test, the doctor inserts an endoscope—a long, flexible, lighted tube connected to a computer and TV monitor—into the anus to see the inside of the colon and rectum. The doctor will be able to see any inflammation, bleeding, or ulcers on the colon wall. During the exam, the doctor may do a biopsy, which involves taking a sample of tissue from the lining of the colon to view with a microscope. A barium enema x-ray of the colon may also be required. This procedure involves filling the colon with barium, a chalky white solution. The barium shows up white on x-ray film, allowing the doctor a clear view of the colon, including any ulcers or other abnormalities that might be there.

Treatment for ulcerative colitis depends on the seriousness of the disease. Most people are treated with medication. In severe cases, a patient may need surgery to remove the diseased colon. Surgery is the only cure for ulcerative colitis. Some people whose symptoms are triggered by certain foods are able to control the symptoms by avoiding foods that upset their intestines, like highly seasoned foods, raw fruits and vegetables, or milk sugar (lactose). Each person may experience ulcerative colitis differently, so treatment is adjusted for each individual. Emotional and psychological support is important. Some people have remissions—periods when the symptoms go away—that last for months or even years. However, most patients' symptoms eventually return. This changing pattern of the disease means one cannot always tell when a treatment has helped. Some people with ulcerative colitis may need medical care for some time, with regular doctor visits to monitor the condition.

The goal of therapy is to induce and maintain remission, and to improve the quality of life for people with ulcerative colitis. Several types of drugs are available:
Aminosalicylates—drugs that contain 5-aminosalicyclic acid (5-ASA), help control inflammation. Sulfasalazine is a combination of sulfapyridine and 5-ASA and is used to induce and maintain remission. The sulfapyridine component carries the anti-inflammatory 5-ASA to the intestine. However, sulfapyridine may lead to side effects such as include nausea, vomiting, heartburn, diarrhea, and headache. Other 5-ASA agents such as olsalazine, mesalamine, and balsalazide, have a different carrier, offer fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first.
Corticosteroids—such as prednisone and hydrocortisone also reduce inflammation. They may be used by people who have moderate to severe ulcerative colitis or who do not respond to 5-ASA drugs. Corticosteroids, also known as steroids, can be given orally, intravenously, through an enema, or in a suppository, depending on the location of the inflammation. These drugs can cause side effects such as weight gain, acne, facial hair, hypertension, mood swings, and an increased risk of infection. For this reason, they are not recommended for long-term use.
Immunomodulators—such as azathioprine and 6-mercapto-purine (6-MP) reduce inflammation by affecting the immune system. They are used for patients who have not responded to 5-ASAs or corticosteroids or who are dependent on corticosteroids. However, immunomodulators are slow-acting and may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids.
   Other drugs may be given to relax the patient or to relieve pain, diarrhea, or infection.

Occasionally, symptoms are severe enough that the person must be hospitalized. For example, a person may have severe bleeding or severe diarrhea that causes dehydration. In such cases the doctor will try to stop diarrhea and loss of blood, fluids, and mineral salts. The patient may need a special diet, feeding through a vein, medications, or sometimes surgery.

About 25-40% of ulcerative colitis patients must eventually have their colons removed because of massive bleeding, severe illness, rupture of the colon, or risk of cancer. Sometimes the doctor will recommend removing the colon if medical treatment fails or if the side effects of corticosteroids or other drugs threaten the patient's health. Surgery to remove the colon and rectum, known as proctocolectomy, is followed by one of the following:
Ileostomy, in which the surgeon creates a small opening in the abdomen, called a stoma, and attaches the end of the small intestine, called the ileum, to it. Waste will travel through the small intestine and exit the body through the stoma. The stoma is about the size of a quarter and is usually located in the lower right part of the abdomen near the beltline. A pouch is worn over the opening to collect waste, and the patient empties the pouch as needed.
Ileoanal anastomosis, or pull-through operation, which allows the patient to have normal bowel movements because it preserves part of the anus. In this operation, the surgeon removes the diseased part of the colon and the inside of the rectum, leaving the outer muscles of the rectum. The surgeon then attaches the ileum to the inside of the rectum and the anus, creating a pouch. Waste is stored in the pouch and passed through the anus in the usual manner. Bowel movements may be more frequent and watery than before the procedure. Inflammation of the pouch (pouchitis) is a possible complication.
   Not every operation is appropriate for every person. Which surgery to have depends on the severity of the disease and the patient's needs, expectations, and lifestyle. People faced with this decision should get as much information as possible by talking to their doctors, to nurses who work with colon surgery patients (enterostomal therapists), and to other colon surgery patients. Patient advocacy organizations can direct people to support groups and other information resources.

Most people with ulcerative colitis will never need to have surgery. If surgery does become necessary, however, some people find comfort in knowing that after the surgery, the colitis is cured and most people go on to live normal, active lives.

B. Crohn's Disease

As with ulcerative colitis, O-glycans have been suggested as playing a role in Crohn's disease, another inflammatory disease of the gastro-intestinal tract. Crohn's disease is characterized by intestinal inflammation and the development of intestinal stenosis and fistulas; neuropathy often accompanies these symptoms. One hypothesis for the etiology of Crohn's disease is that a failure of the intestinal mucosal barrier, possibly resulting from genetic susceptibilities and environmental factors (e.g., smoking), exposes the immune system to antigens from the intestinal lumen including bacterial and food antigens (e.g., Soderholm et al., 1999; Hollander et al., 1986; Hollander, 1992). Another hypothesis is that persistent intestinal infection by pathogens such as Mycobacterium paratuberculosis, Listeria monocytogenes, abnormal Escherichia coli, or paramyxovirus, stimulates the immune response; or alternatively, symptoms result from a dysregulated immune response to ubiquitous antigens, such as normal intestinal microflora and the metabolites and toxins they produce (Sartor, 1997). The presence of IgA and IgG anti-Sacccharomyces cerevisiae antibodies (ASCA) in the serum was found to be highly diagnostic of pediatric Crohn's disease (Ruemmele et al., 1998; Hoffenberg et al., 1999).

Recent efforts to develop diagnostic and treatment tools against Crohn's disease have focused on the central role of cytokines (Schreiber, 1998; van Hogezand & Verspaget, 1998). Cytokines are small secreted proteins or factors (5 to 20 kD) that have specific effects on cell-to-cell interactions, intercellular communication, or the behavior of other cells. Cytokines are produced by lymphocytes, especially T_{H}1 and T_{H}2 lymphocytes, monocytes, intestinal macrophages, granulocytes, epithelial cells, and fibroblasts (reviewed in Rogler &. Andus, 1998; Galley & Webster, 1996). Some cytokines are pro-inflammatory (e.g., TNF-α, IL-1 (α and β), IL-6, IL-8, IL-12, or leukemia inhibitory factor (LIF)); others are anti-inflammatory (e.g., IL-1 receptor antagonist, IL-4, IL-10, IL-11, and TGF-β). However, there may be overlap and functional redundancy in their effects under certain inflammatory conditions.

In active cases of Crohn's disease, elevated concentrations of TNF-α and IL-6 are secreted into the blood circulation, and TNF-α, IL-1, IL-6, and IL-8 are produced in excess locally by mucosal cells (id.; Funakoshi et al., 1998). These cytokines can have far-ranging effects on physiological systems including bone development, hematopoiesis, and liver, thyroid, and neuropsychiatric function. Also, an imbalance of the IL-1β/IL-1ra ratio, in favor of pro-inflammatory IL-1β, has been observed in patients with Crohn's disease (Rogler & Andus, 1998; Saiki et al., 1998; Dionne et al., 1998; but see Kuboyama, 1998). One study suggested that cytokine profiles in stool samples could be a useful diagnostic tool for Crohn's disease (Saiki et al., 1998).

Anti-inflammatory drugs, such as 5-aminosalicylates (e.g., mesalamine) or corticosteroids, are typically prescribed, but are not always effective (reviewed in Botoman et al., 1998). Immunosuppression with cyclosporine is sometimes beneficial for patients resistant to or intolerant of corticosteroids (Brynskov et al., 1989). In Crohn's disease, a dysregulated immune response is skewed toward cell-mediated immunopathology (Murch, 1998). But immunosuppressive drugs, such as cyclosporine, tacrolimus, and mesalamine have been used to treat corticosteroid-resistant cases of Crohn's disease with mixed success (Brynskov et al., 1989; Fellerman et al, 1998). Nevertheless, surgical correction is eventually required in 90% of patients; 50% undergo colonic resection (Leiper et al., 1998; Makowiec et al., 1998). The recurrence rate after surgery is high, with 50% requiring further surgery within 5 years (Leiper et al., 1998; Besnard et al., 1998). Other therapies include the use of various cytokine antagonists (e.g., IL-1ra), inhibitors (e.g., of IL-1β converting enzyme and antioxidants) and anti-cytokine antibodies (Rogler and Andus, 1998; van Hogezand & Verspaget, 1998; Reimund et al, 1998; Lugering et al., 1998; McAlindon et al., 1998). Monoclonal antibodies against TNF-α have been tried with some success in the treatment of Crohn's disease (Targan et al., 1997; Stack et al., 1997; van Dullemen et al, 1995).

Another approach to the treatment of Crohn's disease has focused on at least partially eradicating the bacterial community that may be triggering the inflammatory response and replacing it with a non-pathogenic community. For example, U.S. Pat. No. 5,599,795 discloses a method for the prevention and treatment of Crohn's disease in human patients. Their method was directed to sterilizing the intestinal tract with at least one antibiotic and at least one anti-fungal agent to kill off the existing flora and replacing them with different, select, well-characterized bacteria taken from normal humans. Borody taught a method of treating Crohn's disease by at least partial removal of the existing intestinal microflora by lavage and replacement with a new bacterial community introduced by fecal inoculum from a disease-screened human donor or by a composition comprising Bacteroides and Escherichia coli species. (U.S. Pat. No. 5,443,826). However, there has been no known cause of Crohn's disease to which diagnosis and/or treatment could be directed.

### III. DIAGNOSTIC METHODS

### A. Nucleic Acid Based Diagnostics

Certain embodiments of the present invention concern the analysis of nucleic acids encoding molecules (e.g., enzymes, chaperones) involved in O-glycan synthesis, processing and trafficking. Such targets include T-synthase (NM_{—}020156), C3Gnt (NM_{—}006577) and Cosmc (NM_{—}001011551), and the analysis may range from looking for gross genetic aberrations such as large deletions or gene truncations, to very discrete changes including amino acid substitutions and single nucleotide polymorphisms.

Many methods described herein will involve the use of amplification primers, oligonucleotide probes, and other nucleic acid elements involved in the analysis of genomic DNA, cDNA or mRNA transcripts. The term “nucleic acid” is well known in the art. A “nucleic acid” as used herein will generally refer to a molecule (i.e., a strand) of DNA or RNA comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product.

These definitions generally refer to a single-stranded molecule, but in specific embodiments will also encompass an additional strand that is partially, substantially or fully complementary to the single-stranded molecule. Thus, a nucleic acid may encompass a double-stranded molecule that comprises complementary strands or “complements” of a particular sequence comprising a molecule. In particular aspects, a nucleic acid encodes a protein or polypeptide, or a portion thereof.

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference). In some aspects, a nucleic acid is a pharmacologically acceptable nucleic acid. Pharmacologically acceptable compositions are known to those of skill in the art, and are described herein.

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Complements

As discussed above, the present invention encompasses a nucleic acid that is complementary to a nucleic acid. A nucleic acid is “complements” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

4. Nucleic Acid Detection and Evaluation

Those in the art will readily recognize that nucleic acid molecules may be double-stranded molecules and that reference to a particular site on one strand refers, as well, to the corresponding site on a complementary strand. Thus, in defining a polymorphic site, reference to an adenine, a thymine (uridine), a cytosine, or a guanine at a particular site on the plus (sense or coding) strand of a nucleic acid molecule is also intended to include the thymine (uridine), adenine, guanine, or cytosine (respectively) at the corresponding site on a minus (antisense or noncoding) strand of a complementary strand of a nucleic acid molecule. Thus, reference may be made to either strand and still comprise the same polymorphic site and an oligonucleotide may be designed to hybridize to either strand. Throughout the text, in identifying a polymorphic site, reference is made to the sense strand, only for the purpose of convenience.

Typically, the nucleic acid mixture is isolated from a biological sample taken from the individual, such as a blood, fecal or tissue (e.g., intestinal mucosal) sample using standard techniques such as disclosed in Jones (1963) which is hereby incorporated by reference. Other suitable tissue samples include whole blood, saliva, tears, urine, sweat, buccal, skin and hair. The nucleic acid mixture may be comprised of genomic DNA, mRNA, or cDNA and, in the latter two cases, the biological sample must be obtained from an organ in which the SOD1 gene is expressed. Furthermore it will be understood by the skilled artisan that mRNA or cDNA preparations would not be used to detect polymorphisms located in introns or in 5′ and 3′ non-transcribed regions.

The identity of a nucleotide (or nucleotide pair) at a polymorphic site may be determined by amplifying a target region(s) containing the polymorphic site(s) directly from one or both copies of the gene present in the individual and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

The target region(s) may be amplified using any oligonucleotide-directed amplification method, including but not limited to polymerase chain reaction (PCR) (U.S. Pat. No. 4,965,188), ligase chain reaction (LCR) (Barany et al., 1991; WO90/01069), and oligonucleotide ligation assay (OLA) (Landegren et al., 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766; WO89/06700) and isothermal methods (Walker et al., 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype for one or more polymorphic sites in the gene of an individual may also be determined by hybridization of one or both copies of the gene, or a fragment thereof, to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., 1985; Meyers et al., 1985) and proteins which recognize nucleotide mismatches, such as the E. coli mutS protein (Modrich, 1991). Alternatively, variant alleles can be identified by single strand conformation polymorphism (SSCP) analysis (Orita et al., 1989; Humphries, et al., 1996) or denaturing gradient gel electrophoresis (DGGE) (Wartell et al., 1990; Sheffield et al., 1989).

A polymerase-mediated primer extension method may also be used to identify the polymorphism(s). Several such methods have been described in the patent and scientific literature. Extended primers containing a polymorphism may be detected by mass spectrometry as described in U.S. Pat. No. 5,605,798. Another primer extension method is allele-specific PCR (Ruano et al., 1989; Ruano et al., 1991; WO 93/22456; Turki et al, 1995).

a. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kB or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the SOD1 gene locus, variants and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (DGGE), restriction fragment length polymorphism analysis (RFLP), chemical or enzymatic cleavage methods, direct sequencing of target regions amplified by PCR™ (see above), single-strand conformation polymorphism analysis (SSCP) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

e. Polymorphic Nucleic Acid Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms is generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

Several methods have been developed to screen polymorphisms and some examples are listed below. The reference of Kwok and Chen (2003) and Kwok (2001) provide overviews of some of these methods; both of these references are specifically incorporated by reference. SNPs can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

i. DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

ii. Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′- to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

iii. Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al, 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

iv. Extension in Solution

French Patent 2,650,840 and PCT Application WO91/02087 discuss a solution-based method for determining the identity of the nucleotide of a polymorphic site. According to these methods, a primer complementary to allelic sequences immediately 3′- to a polymorphic site is used. The identity of the nucleotide of that site is determined using labeled dideoxynucleotide derivatives which are incorporated at the end of the primer if complementary to the nucleotide of the polymorphic site.

v. Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

vi. Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here, PCR is used to achieve the exponential amplification of target DNA, which is then detected using the OLA.

vii. Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

viii. Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al. (2004). In some cases, the assay is conducted on a solid-surface or in an array format.

### B. O-Glycan Based Diagnostics

The present invention also contemplates the examination of protein or carbohydrate structure, protein or carbohydrate level, or protein activity for diagnostic/prognostic methods. Such examination can be by various methods including antibody-based assays for O-glycan expression or structure (Western blots, ELISA), gel electrophoresis, chromatographic separation (HPLC), or mass spectroscopy of proteins or O-glycans, or assays for expression or activity of enzymes that modulate O-glycan synthesis, processing or trafficking. In addition, it is also possible to identify endogenous antibodies with unique specificity profiles that indicate presence of altered O-glycans. Various of these techniques are discussed below.

1. Protein Purification

It may be desirable to purify proteins from a sample, including antibodies. Such techniques are widely employed and the invention is not intended to be limited with respect to protein purification. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention may concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

A variety of techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

2. Antibodies and Lectins

Another embodiment of the present invention involves the use of carboydrate binding agents, such as antibodies and lectins. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Harlow et al., 1988; incorporated herein by reference).

Monoclonal antibodies against the T, Tn, or sialyl-Tn antigens (mouse IgG or IgM) are available and may be used in any of the assays described below.

a. Antibody Generation

In certain embodiments, the present invention involves antibodies, in particular those that recognize various O-glycan structures. In addition to antibodies generated against full length proteins, antibodies also may be generated in response to smaller constructs comprising epitopic core regions. The techniques for preparing and using various antibody-based constructs and fragments are well known in the art. Means for preparing and characterizing antibodies are also well known in the art (see, e.g., Harlow and Lane, 1988; incorporated herein by reference).

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

b. Immunodetection Methods

As discussed, in some embodiments, the present invention concerns immunodetection methods for binding, purifying, removing, determining, and/or otherwise detecting biological components such as antigenic regions on proteins and O-glycans. Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al., 1999; Gulbis et al, 1993; De Jager et al., 1993; and Nakamura et al., 1987, each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR: reaction with suitable primers with appropriate controls. At least in theory, the enormous amplification capability and specificity of PCR can be utilized to detect a single antigen molecule.

i. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label. The ELISA may be based on differential binding of an antibody to a protein with Arg389 versus Gly389.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

ii. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize Fortilin or to evaluate the amount Fortilin in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

c. Lectins

Lectins are sugar-binding proteins that are highly specific for particular sugar moieties. They typically play a role in biological recognition phenomena involving cells and proteins. For example, some bacteria use lectins to attach themselves to the cells of the host organism during infection. The are also involved in normal processes such as cell adhesion.

Helipomatia agglutinin (HPA) may be used for the detection of the Tn antigen, and Artocarpus polyphemus lectin (Jacalin lectin) and Arachis hypogaea (peanut) agglutinin (PNA) can be used for detection of T antigen (core 1 O-glycan). For sialic acid, the inventors contemplate the use of Maackia amurensis hemagglutinin (MAH), which recognizes a2-3-linked SA, and Sambucus nigra agglutinin (SNA), which recognizes a2-6-linked sialic acid. Biotinylated or horseradish peroxidase (HRP)-conjugated forms of the lectins are commercially available (EY Laboratories and Vector Laboratories). Lectin-binding assays will follow the general format of immunodetection methods, set forth above, and are known to those of skill in the art.

### IV. THERAPIES

In addition to diagnosing diseases, the present invention also permits earlier therapeutic, and even prophylatic intervention, with respect to patients having early stage IBD or pre-clinical conditions that could lead to IBD. In addition, the present invention permits the monitoring of a therapy to determine its efficacy. Therapeutic approaches for IBD are therefore discussed below.

A. Treatments for Inflammatory Bowel Diseases

1. Aminosalicylates

Aminosalicylates are anti-inflammatory drugs in the aspirin family. There are five aminosalicylate preparations available for use in the United States: sulfasalazine (Azulfidine), mesalamine (Asacol, Pentasa), olsalazine (Dipentum), and balsalazide (Colazal). These drugs can be given either orally or rectally (enema, suppository formulations).

2. NSAIDS

Nonsteroidal anti-inflammatory agents (NSAIDs) work by inhibiting the production of prostaglandins. Non-limiting examples include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate.

3. Corticosteroids

Corticosteroids are powerful, fast-acting anti-inflammatory agents. Their use in IBD is for acute flare-ups only. Corticosteroids may be administered by a variety of routes, depending upon the location and severity of disease; they may be administered intravenously (methylprednisolone, hydrocortisone) in the hospital, orally (prednisone, prednisolone, budesonide, dexamethasone), or rectally (enema, suppository, foam preparations). Corticosteroids tend to provide rapid relief of symptoms as well as a significant decrease in inflammation, but their side effects limit their use (particularly longer-term use).

4. Immune Modifiers

Immune modifiers include 6-mercaptopurine (6-MP, Purinethol) and azathioprine (Imuran). Immune modifiers may work by causing a reduction in the lymphocyte count (a type of white blood cell). They are often used when aminosalicylates and corticosteroids are either ineffective or only partially effective. They are useful in reducing or eliminating some patient's dependence on corticosteroids. Immune modifiers may also prove helpful in maintaining remission in some persons with refractory ulcerative colitis.

5. Anti-TNF Agents

Infliximab (Remicade) is an anti-TNF agent, acting by binding to TNF, thereby inhibiting its effects on the tissues. It is approved by the FDA for the treatment of persons with moderate-to-severe Crohn's Disease who have had an inadequate response to standard medications. In such persons, a response rate of 80% and a remission rate of 50% have been reported.

6. Antibiotics

Metronidazole and ciprofloxacin are the most commonly used antibiotics in persons with IBD. Antibiotics are used sparingly in persons with ulcerative colitis because they have an increased risk of developing antibiotic-associated pseudomembranous colitis. In persons with Crohn's Disease, antibiotics are used for the treatment of complications (perianal disease, fistulae, inflammatory mass).

7. Symptomatic Treatments

One can also provide antidiarrheal agents, antispasmodics, and acid suppressants for symptomatic relief.

8. O-glycan Compositions

O-glycan compositions, in one aspect, relate to mucins. Mucins are high-molecular weight epithelial glycoproteins with a high content of clustered oligosaccharides O-glycosidically linked to tandem repeat peptides rich in threonine, serine, and proline. There are two structurally and functionally distinct classes of mucins: secreted gel-forming mucins (MUC2, MUC5AC, MUC5B, and MUC6) and transmembrane mucins (MUC1, MUC3A, MUC3B, MUC4, MUC 12, MUC 17), although the products of some MUC genes do not fit well into either class (MUC7, MUC8, MUC9, MUC13, MUC15, MUC16). The O-glycosidically linked oligosaccharides of mucins can be described in terms of core type, backbone type, and peripheral structures. As discussed in PCT/US2007/065971, such compositions can find therapeutic use in treating IBD.

B. Combination Therapies

Any of the foregoing treatments may be combined for the treatment of an inflammatory bowel disease. By combining agents, an additive effect may be achieved while not increasing the toxicity (if any) associated with a monotherapy. In addition, it is possible that more than additive effects (“synergism”) may be observed. Thus, combination therapies are a common way to exploit new therapeutic regimens.

Any one treatment may precede, be co-current with and/or follow the other agent(s) by intervals ranging from minutes to weeks. In embodiments where the various agent(s) are applied separately to a cell, tissue or organism, one would generally ensure that a significant period of time did not expire between the time of each delivery, such that both agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute). In other aspects, an agent may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the other treatment.

Various combination regimens of one or more agents may be employed. Non-limiting examples of such combinations are shown below, wherein a first agent or treatment is “A” and a second agent or treatment is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Traditionally, a stepwise approach to treatment of IBD is employed. The aminosalicylates/NSAIDS and symptomatic agents would be considered the first line of treatment. Next, one would turn to antibiotics particularly in persons with Crohn's Disease who have perianal disease or an inflammatory mass. Corticosteroids are the next line of defense, having more significant side effects that other anti-inflammatories. Immune modifying agents can be used if corticosteroids do not provide the desired results. Drugs from any of the foregoing categories may be used together. Some specific examples are provided below.

### C. Pharmaceutical Compositions

Pharmaceutical compositions of the present invention comprise an effective amount of an agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered orally, or rectally, but may also be administered intratracheally, intranasally, subcutaneously, mucosally, locally, inhalation (e.g. aerosol inhalation), injection, infusion, continuous infusion, localized perfusion bathing target cells directly, via a catheter, via a lavage, or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In particular embodiments, the therapeutic compositions of the present invention are prepared for administration by such routes as oral ingestion. In these embodiments, the solid composition may comprise, for example, solutions, suspensions, emulsions, tablets, pills, capsules (e.g., hard or soft shelled gelatin capsules), delayed release capsules, sustained release formulations, buccal compositions, troches, elixirs, suspensions, syrups, wafers, or combinations thereof. Oral compositions may be incorporated directly with the food of the diet. Specific carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain specific embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum. After insertion, suppositories soften, melt or dissolve in the cavity. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A. Introduction

Ulcerative colitis is a common form of inflammatory bowel diseases (IBD). It has been reported that the expression of O-glycans, which are an essential part of the intestinal mucus layer, was altered abnormally in patients with ulcerative colitis. Tn antigen is a biosynthetic intermediate in the formation of mucin-type O-glycans. Abnormal expression of Tn antigen is often reported to be associated with human cancers, including colorectal carcinomas. The inventor's lab engineered mice lacking intestinal epithelial O-glycans (Epi T-syn^{-/-} mice). Epi Tsyn^{-/-} mice express Tn antigen in the intestinal epithelium and develop spontaneous colitis that resembles human colitis. These results indicate intestinal O-glycans play an important role in the pathogenesis of colitis. The aim of this study is to examine the expression of Tn antigen in colon lavages from patients with colitis, and investigate whether Tn antigen expression is associated with colitis in human.

### B. Materials

Samples. The samples of human colon ravages were provided by a collaborator. They are from 6 patients. Each sample was labeled as PBS+TX and V9, respectively. PBS+TX (totally 100 μl volume) are processed samples (initial extracts acetone precipitated followed by extraction of pellet with 9M urea+2% CHAPS). V9 are unprocessed samples (totally 25 μl volume).

Probes for glycan detection. Biotinlyated lectin of Helix pomatia agglutinin (HPA) is a commercial reagent, which recognizes the non-sialylated Tn antigen (EY Laboratories Inc). Anti-Tn IgM mAb (BAG-6) was used to detect Tn antigen expression.

### C. Methods

Due to limited volume of these samples, the inventors were unable to measure protein concentrations, and thus used different volumes of samples. Samples were reduced with 2-mercaptoethanol, followed by boiling for 10 minutes, and then run on 4˜15% gradient gel for 2 hrs at 150 v.

PAS (periodic acid-Schiff's reagent) staining. The inventors used periodic acid-Schiff's reagent (PAS) to determine the expression of intestinal neutral carbohydrate, according to the protocol.

Immunoblot. After electrophoresis, gels were transferred to PVDF membranes for 1 hr at 15 v. The membranes were blocked with 3% BSA in TBS-0.1% Tween20 for 1 hr at RT, and subsequently incubated with HPA (10 μg/ml) or anti-Tn IgM antibody (1 μg/ml) in TBST at 4° C. overnight. After washing by 15 min×3 times, the membranes were incubated with strep HRP (1:4000) or HRP-conjugated goat anti-mouse IgM (1:4000) for 2 hrs at RT. Finally, the membranes were developed with ECL kit from Amersham.

### D. Results

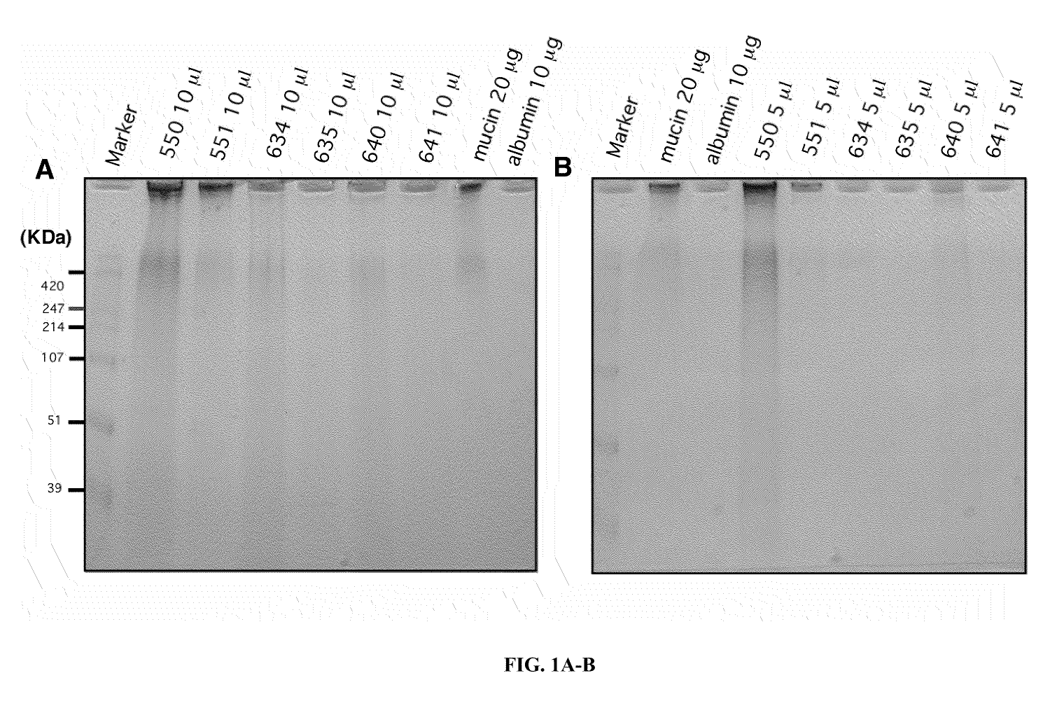

PAS staining shows all samples contain carbohydrate structures. The inventors first used PAS to stain neutral carbohydrates in these human colon lavages. The results demonstrated that in all samples had positive staining of PAS. Staining of processed samples was stronger than unprocessed samples, suggesting that processed samples have higher concentration of carbohydrate structures. Among all samples, 550 appears to have the highest sugar staining (FIGS. 1A-B)

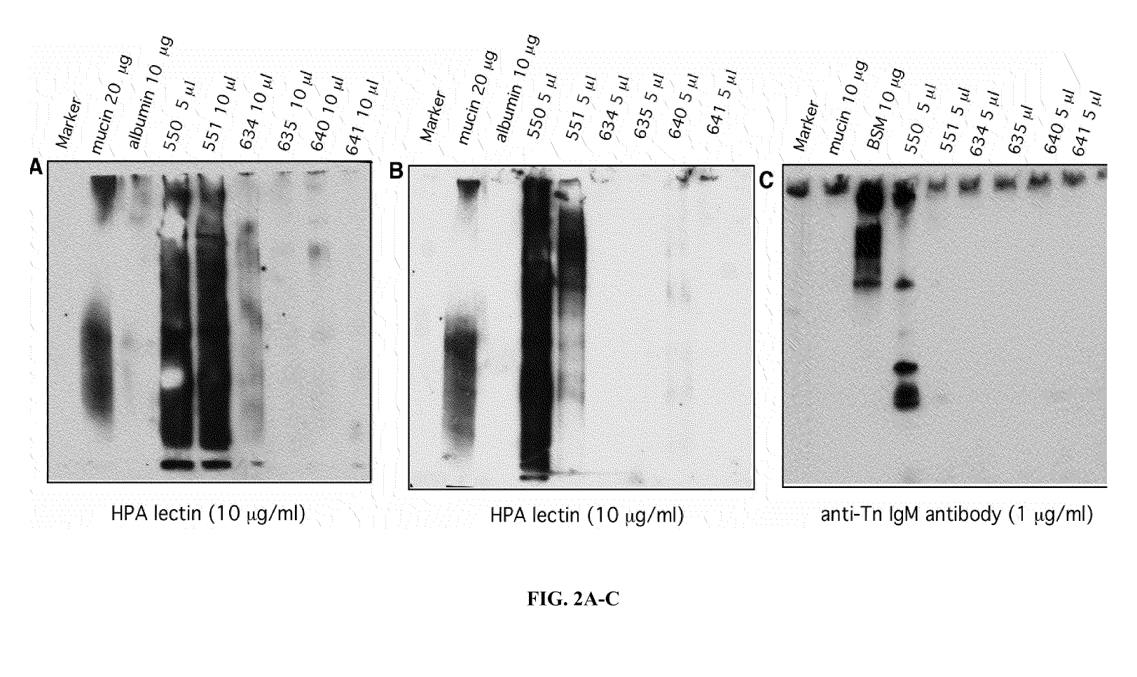

Samples 550 and 551 are Tn positive. The inventors further examined the expression of Tn antigen in these samples. Firstly, they used HPA, a lectin that recognizes the nonsialylated Tn antigen, to detect the expression of Tn antigen. Samples 550 and 551 were reactive to HPA (FIGS. 2A-B). To confirm this result, they employed anti-Tn IgM antibody. Consistent with HPA staining, samples 550 and 551 bound to anti-Tn antibody (FIG. 2C). These results indicated both 550 and 551 express Tn antigens. Both 550 and 551 were collected from patients with active colitis.

### Example 2

### A. Materials

Samples. Colon tissue specimens that meet selection criteria for the individual patient cohorts at the OU Medical Center were subjected to immunohistochemical analyses for Tn antigens. A total of 25 samples were collected and processed according the protocol. A total of 15 samples from patients with ulcerative colitis were chosen. A total of 10 samples from patients with Crohn's colitis patients were chosen. A total of 10 normal colons were selected as the control. 5 samples from non-IBD patients with idiopathic diarrhea were included as acute non-IBD controls.

Tn staining of paraffin embedded tissues. Deparaffinized tissue sections were treated with neuraminidase (0.5 U/ml in 10 mM Tris, pH 6.3) at 37° C. for 2-3 hours. After washing, the sections were incubated with biotinated anti-Tn antibody (1:100 using 1% BSA in PBS) for 45 minutes at RT followed by Streptavidin/HRP (1:500 using 1% BSA in PBS) for 30 minutes at RT. The slides were developed with DAB substrate (Vector) for peroxidase (approximately 2-10 minutes), and conterstained with Hematoxylin.

### B. Results

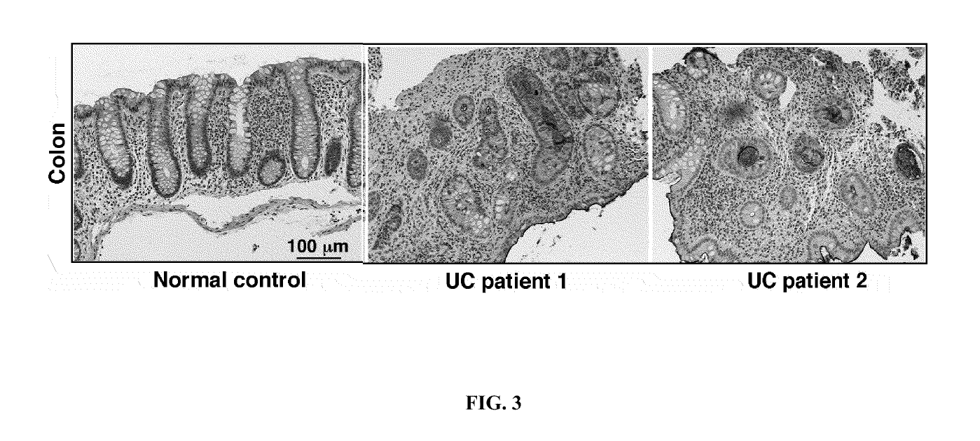

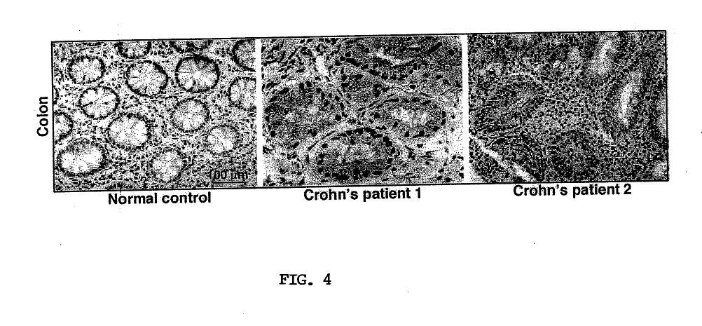

Epithelial cells of five out of the fifteen ulcerative colitis samples, and three out of ten Crohn's samples, were Tn positive (FIGS. 3-4), while all ten samples from non-colitis patients were negative. Approximately 50% of the biopsy samples were either too small or did not have epithelium as a result of inflammation. Therefore, the rate of Tn detection may be an underestimate. Interestingly, there were Tn-positive and negative crypts in a single colonic section of a patient with colitis (FIG. 3, UC patient #2), suggesting clonal abnormality of O-glycosylation. Due to the limited samples size, the inventor did not observe any significant association of Tn expression with disease activities.

### C. Conclusions

Tn expression is associated with both colitis and Crohn's disease. Due to sample size limitation, no significant association has been found between Tn expression and disease activities of colitis and Crohn's disease.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 5,440,013
U.S. Pat. No. 5,443,826
U.S. Pat. No. 5,446,128
U.S. Pat. No. 5,475,085
U.S. Pat. No. 5,599,795
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,840,833
U.S. Pat. No. 5,859,184
U.S. Pat. No. 5,929,237
Besnard et al., Gut, 43(5):634-638, 1998.
Botoman et al., Am. Fam. Physician, 57(1):57-68, 1998.
Brynskov et al., N. Engl. J. Med., 321(13):845-850, 1989.
Byrd and Bresalier, Cancer Metastasis Rev., 23(1-2):77-99, 2004.
Corfield et al., Front Biosci., 6:D1321-1357, 2001.
Dionne et al., Clin. Exp. Immunol., 112(3):435-442, 1998.
Dixon et al., Postgrad Med J., 82(963):76-78, 2006.
Elson et al., Inflamm. Bowel Dis., 9:137-153, 2003.
Fellerman et al., Am. J. Gastroenterol., 93(10):1860-1866, 1998.
Feste et al., J. Chromatogr., 518:349-359, 1990.
Funakoshi et al., Digestion, 59(1):73-78, 1998.
Galley and Webster, Br. J. Anaesth., 77:11-16, 1996.
Hoffenberg et al., J. Pediatr., 134(4):447-452, 1999.
Hollander et al., Ann. Intern. Med., 105:883-885, 1986.
Hollander, Scand. J. Gastroenterol., 27:721-726, 1992.
Iwai et al., J. Biol. Chem., 277:12802-12809, 2002.
Johannesson et al., J. Med. Chem., 42(4):601-608, 1999.
Johnson et al., In: Biotechnology And Pharmacy, Pezzuto et al. (Eds.), Chapman and Hall, NY, 1993.
Kuboyama, Kurume Med. J., 45(1):33-37, 1998.
Kyte and Doolittle, J. Mol. Biol., 157(1):105-132, 1982.
Leiper et al., Baillieres Clin. Gastroenterol., 12(1):179-199, 1998.
Lugering et al., Ital. J. Gastroenterol. Hepatol., 30(3):338-344, 1998.
Makowiec et al., Z. Gastroenterol., 36(8):619-624, 1998.
McAlindon et al., Gut, 42(2):214-219, 1998.
Murch, Nutrition, 14:780-783, 1998.
Podolsky and Fournier, Gastroenterology, 95:379-387, 1988.
Podolsky Isselbacher, Gastroenterology, 87:991-998, 1984.
Podolsky, N. Engl. J. Med., 347:417-429, 2002.
Reimund et al., Eur. J. Clin. Invest., 28(2):145-150, 1998.
Remington's Pharmaceutical Sciences, 18th Ed. Mack Printing Company, pp. 1289-1329, 1990.
Rhodes, Gut., 40:807-808, 1997.
Rhodes, Lancet., 347:40-44, 1996.
Rogler and Andus, World J. Surg., 22(4):382-389, 1998.
Ruemmele et al., Gastroenterol., 115(4):822-829, 1998.
Saiki et al., Scand. J. Gastroenterol., 33(6):616-622, 1998.
Sartor, Am. J. Gastroenterol., 92(12):5S-11S, 1997.
Sartor, Curr. Opin. Gastroenterol., 19:358-365, 2003.
Schreiber, Neth. J. Med., 53(6):S24-31, 1998.
Soderholm et al., Gastroenterol., 117:65-72, 1999.
Stack et al., Lancet, 349(9051):521-524, 1997.
Stevceva et al., BMC Clin. Pathol., 1:3, 2001.
Targan et al., N. Engl. J. Med., 337(15):1029-1035, 1997.
van IDullemen et al., Gastroenterol., 109(1):129-135, 1995.
van Hogezand and Verspaget, Drugs, 56(3):299-305, 1998.
Varki et al., In: Essentials of Glycobiology, Cold Spring Harbor Press, Cold Spring Harbor, N.Y., 1999.
Vita et al., Biopolymers, 47:93-100, 1998.
Weisshoff et al., Eur. J. Biochem., 259(3):776-788, 1999.
Xia et al., Glycobiology, 15:747-775, 2005.

1. A method of diagnosing or predicting inflammatory bowel disease (IBD) in a subject comprising:
(a) obtaining a sample comprising mucins from said subject;
(b) assessing mucin-related O-glycan structure and/or expression in said sample;
(c) diagnosing or predicting IBD in said subject where mucin-related O-glycan structure or expression is abnormal.

2. The method of claim 1, wherein the sample is a colon lavage, a fecal sample, a colorectal swab, or a colon tissue sample.

3. (canceled)

4. The method of claim 1, wherein O-glycan structure is assessed.

5. The method of claim 4, wherein the O-glycan structure is a core-1 glycan structure.

6. The method of claim 4, wherein the O-glycan structure is a core-3 glycan structure.

7. The method of claim 4, wherein the O-glycan structure is core-1 and core-3 glycan structures.

8. The method of claim 1, wherein O-glycan expression is assessed.

9. The method of claim 1, wherein assessing comprises immunological recognition of T, Tn, sialyl-T and/or sialyl-Tn.

10. (canceled)

11. The method of claim 1, wherein assessing comprises lectin or antibody array analysis.

12. (canceled)

13. The method of claim 1, further comprising obtaining other diagnostic information from said subject.

14. (canceled)

15. A method of diagnosing or predicting inflammatory bowel disease (IBD) in a subject comprising:
(a) obtaining a tissue sample from said subject;
(b) assessing the structure and/or transcription of one or more genes involved in mucin-related O-glycan trafficking, stability, synthesis or processing in a cell of said sample;
(c) diagnosing or predicting IBD in said subject where one or more of said genes involved in mucin-related O-glycan trafficking, stability synthesis or processing is altered in structure or transcription.

16. The method of claim 15, wherein the tissue sample is a colon tissue sample.

17. The method of claim 15, wherein the tissue sample comprises endothelial cells, epithelial cells and/or hematopoietic cells.

18. The method of claim 15, wherein the gene encodes a protein involved in mucin-related O-glycan synthesis.

19. (canceled)

20. The method of claim 15, wherein the gene encodes a protein involved in mucin-related O-glycan processing.

21. The method of claim 15, wherein the gene encodes a protein involved in mucin-related O-glycan trafficking or stability

22-24. (canceled)

25. The method of claim 15, further comprising obtaining other diagnostic information from said subject.

26. (canceled)

27. A method of diagnosing or predicting inflammatory bowel disease (IBD) in a subject comprising:
(a) obtaining a tissue sample from said subject;
(b) assessing the activity or level of one or more proteins involved in mucin-related O-glycan trafficking, stability, synthesis or processing in a cell of said sample;
(c) diagnosing or predicting IBD in said subject where one or more of said genes involved in mucin-related O-glycan trafficking, stability synthesis or processing is altered in activity or level.

28. The method of claim 27, wherein the tissue sample is a colon tissue sample.

29. The method of claim 27, wherein the tissue sample comprises endothelial cells, epithelial cells and/or hematopoietic cells.

30. The method of claim 27, wherein the protein may be involved in mucin-related O-glycan synthesis.

31. (canceled)

32. The method of claim 27, wherein the protein may be involved in mucin-related O-glycan processing.

33. The method of claim 27, wherein the protein may be involved in mucin-related O-glycan trafficking or stability.

34-36. (canceled)

37. The method of claim 27, further comprising obtaining other diagnostic information from said subject.

38. (canceled)

39. A method of diagnosing or predicting inflammatory bowel disease (IBD) in a subject comprising:
(a) obtaining a sample comprising antibodies from said subject;
(b) assessing O-glycan-binding antibodies in said sample;
(c) diagnosing or predicting IBD in said subject where O-glycan-binding antibodies in said sample are abnormal.

40. The method of claim 39, wherein said sample is serum.

41. The method of claim 39, wherein assessing comprise measuring antibody level.

42. The method of claim 39, wherein assessing comprises measuring antibody specificity.

43. The method of claim 39, wherein assessing comprises measure antibody level and specificity.

44-45. (canceled)

46. The method of claim 39, further comprising obtaining other diagnostic information from said subject.

47. (canceled)

48. A method of classifying inflammatory bowel disease (IBD) in a subject comprising:
(a) obtaining a sample comprising mucins from said subject;
(b) assessing mucin-related O-glycan structure and/or expression in said sample;
(c) classifying the IBD in said subject based on the amount of mucin-related O-glycan expression, as compared to a normal and/or abnormal standard.

49-54. (canceled)

55. A method of monitoring disease progression or therapeutic efficacy in a subject with inflammatory bowel disease (IBD) comprising:
(a) obtaining a first sample comprising mucins from said subject;
(b) assessing mucin-related O-glycan structure and/or expression in said first sample;
(c) obtaining a second sample comprising mucins from said subject at a later point in time as compared to said first sample;
(d) assessing mucin-related O-glycan structure and/or expression in said second sample;
(e) comparing mucin-related O-glycan structure and/or expression between said first and second samples.
